(19) United States
(12) Patent Application Publication
(10) Pub. No.: US 2026/0114846 A1
(43) Pub. Date: Apr. 30, 2026

(54) ULTRASOUND IMAGE PROCESSING DEVICE, ULTRASOUND DIAGNOSTIC APPARATUS, AND ULTRASOUND IMAGE PROCESSING PROGRAM

(71) Applicant: FUJIFILM Corporation, Tokyo (JP)

(72) Inventor: Suguru ISHIGURO, Tokyo (JP)

(73) Assignee: FUJIFILM Corporation, Tokyo (JP)

(21) Appl. No.: 19/365,150

(22) Filed: Oct. 21, 2025

(30) Foreign Application Priority Data
Oct. 25, 2024 (JP) 2024-188230

(51) Int. Cl. A61B 8/00; A61B 8/08

(52) U.S. Cl. CPC A61B 8/4254; A61B 8/085; A61B 8/463; A61B 8/5207; A61B 8/5246

(57) ABSTRACT

A processor provided in an ultrasound image processing device is configured to: generate ultrasound image data corresponding to each of ultrasound probes based on a reception signal output from each of the ultrasound probes; acquire position-and-posture information of each of the ultrasound probes; and generate display image data showing an ultrasound image obtained by one of the ultrasound probes and a probe indicator indicating an ultrasound transmission range of the other ultrasound probe, based on the ultrasound image data corresponding to each of the ultrasound probes and the position-and-posture information.

This application claims the priority benefit of Japan application serial no. 2024-188230, filed on October 25, 2024. The entirety of the above-mentioned patent application is hereby incorporated by reference herein and made a part of this specification.

### BACKGROUND OF THE INVENTION

### 1. Field of the Invention

The present disclosure relates to an ultrasound image processing device, an ultrasound diagnostic apparatus, and an ultrasound image processing program, and particularly to a device and a program that generate ultrasound image data corresponding to each ultrasound probe using a plurality of ultrasound probes.

### 2. Description of the Related Art

An ultrasound diagnostic apparatus that can observe a subject from different directions using two or more ultrasound probes has been known. It has been proposed to perform prostate surgery while observing two B-mode images obtained from a rectum and a body surface by, for example, a transrectal probe and an intraoperative probe using such an ultrasound diagnostic apparatus.

In addition, JP2018-20109A discloses an ultrasound diagnostic apparatus that detects a position of an ultrasound probe as a device related to the present disclosure described later.

### SUMMARY OF THE INVENTION

In a case in which the subject is observed from different directions, there is a problem that it is difficult to understand a positional relationship of cross sections in which the B-mode images are observed using each of the ultrasound probes simply by displaying two or more ultrasound images acquired by two or more ultrasound probes.

An object of the present disclosure is to facilitate observation of a subject by an ultrasound diagnostic apparatus using a plurality of ultrasound probes.

The present disclosure relates to an ultrasound image processing device comprising: a processor configured to: generate ultrasound image data corresponding to each of a plurality of ultrasound probes based on a reception signal output from each of the ultrasound probes; acquire position-and-posture information of each of the ultrasound probes; and generate display image data showing an ultrasound image obtained by one of the plurality of ultrasound probes and a probe indicator indicating an ultrasound transmission range of the other ultrasound probe, based on the ultrasound image data corresponding to each of the ultrasound probes and the position-and-posture information.

In one embodiment, the probe indicator indicates a region in which a scan range of an ultrasound beam formed by a first ultrasound probe, which is one of the plurality of ultrasound probes, and a scan range of an ultrasound beam formed by a second ultrasound probe, which is the other ultrasound probe, intersect each other.

In one embodiment, the probe indicator includes an intersection line between the scan range of the ultrasound beam formed by the first ultrasound probe and the scan range of the ultrasound beam formed by the second ultrasound probe, and an extension line formed by extending the intersection line, and the extension line extends in accordance with a projection image in which the scan range of the ultrasound beam formed by the second ultrasound probe is projected onto an ultrasound image obtained by the first ultrasound probe in a direction of the ultrasound beam formed by the second ultrasound probe.

In one embodiment, the display image data is image data in which the probe indicator is displayed in a superimposed manner on any of an ultrasound image obtained by the first ultrasound probe or an ultrasound image obtained by the second ultrasound probe.

In one embodiment, the display image data includes a probe positional relationship image showing a scan range of an ultrasound beam formed by a first ultrasound probe, which is one of the plurality of ultrasound probes, a scan range of an ultrasound beam formed by a second ultrasound probe, which is the other ultrasound probe, and a positional relationship between the first ultrasound probe and the second ultrasound probe.

In one embodiment, each of the ultrasound probes, and a position-and-posture sensor provided in each of the ultrasound probes are provided, and the processor is configured to: generate the position-and-posture information of each of the ultrasound probes based on an output value of the position-and-posture sensor provided in each of the ultrasound probes; and generate the display image data based on the position-and-posture information of one of the plurality of ultrasound probes and the position-and-posture information of the other ultrasound probe.

Further, the present disclosure provides a storage medium storing an ultrasound image processing program read by an ultrasound image processing device, the program causing a processor provided in the ultrasound image processing device to execute a process comprising: generating ultrasound image data corresponding to each of a plurality of ultrasound probes based on a reception signal output from each of the ultrasound probes; acquiring position-and-posture information of each of the ultrasound probes; and generating display image data showing an ultrasound image obtained by one of the plurality of ultrasound probes and a probe indicator indicating an ultrasound transmission range of the other ultrasound probe, based on the ultrasound image data corresponding to each of the ultrasound probes and the position-and-posture information.

According to the present disclosure, it is possible to facilitate the observation of the subject by the ultrasound diagnostic apparatus using the plurality of ultrasound probes.

### BRIEF DESCRIPTION OF THE DRAWINGS

FIG. 1 is a diagram showing a configuration of an ultrasound diagnostic apparatus according to an embodiment of the present disclosure.

FIG. 2 is a diagram showing an example of diagnosis performed using an ultrasound diagnostic apparatus.

FIG. 3 is a diagram showing a probe positional relationship diagram schematically showing an example of a position and a posture of each ultrasound probe.

FIG. 4 is a diagram showing an example of an intersection line/convex scan image displayed on a display unit.

FIG. 5 is a diagram of a first observation plane and a second observation plane, which are taken in a direction perpendicular to a normal line of the first observation plane and toward a plane on which the second observation plane extends.

FIG. 6 is a diagram in which an extension line is added to the probe positional relationship diagram shown in FIG. 3.

FIG. 7 is a diagram showing an example of the intersection line/convex scan image.

FIG. 8 is a diagram showing an example of an intersection line/linear scan image.

FIG. 9 is a diagram showing an example of a three-dimensional probe positional relationship image.

FIG. 10 is a diagram showing the intersection line/convex scan image, the intersection line/linear scan image, and the three-dimensional probe positional relationship image displayed side by side on the display unit.

FIG. 11 is a diagram showing the intersection line/convex scan image and the intersection line/linear scan image displayed side by side on the display unit, and the three-dimensional probe positional relationship image displayed in a superimposed manner on the intersection line/convex scan image.

FIG. 12 is a diagram showing the intersection line/convex scan image, a two-dimensional intersection region/linear scan image, and the three-dimensional probe positional relationship image displayed side by side on the display unit.

### DESCRIPTION OF THE PREFERRED EMBODIMENTS

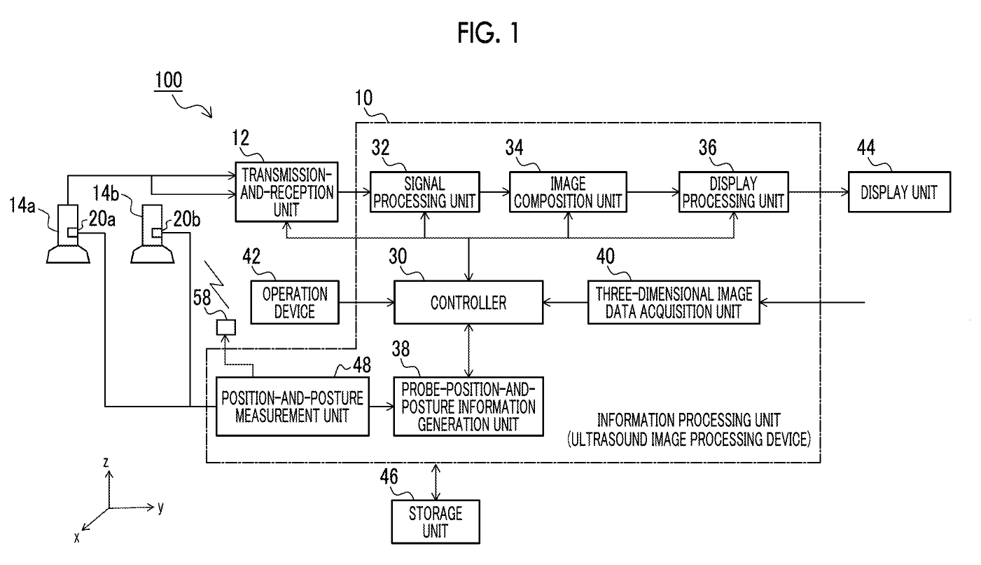

An embodiment of the present disclosure will be described with reference to the accompanying drawings. The same components shown in the plurality of drawings are denoted by the same reference numerals to simplify the description thereof. FIG. 1 shows a configuration of an ultrasound diagnostic apparatus 100 according to the embodiment of the present disclosure. The ultrasound diagnostic apparatus 100 comprises an information processing unit 10, a transmission-and-reception unit 12, an ultrasound probe 14a, an ultrasound probe 14b, an operation device 42, a display unit 44, and a positioning signal generator 58.

A storage unit 46 shown in FIG. 1 together with the ultrasound diagnostic apparatus 100 may be a storage (storage medium) such as a hard disk mounted on the ultrasound diagnostic apparatus 100. In addition, the storage unit 46 may be a memory (storage medium) of a computer on a local area network or a memory of a computer on an electric communication line, such as the Internet.

A part or all of the information processing unit 10 may be configured by, for example, one or more computers that execute a program stored in the storage unit 46. The information processing unit 10 executes the program to configure a controller 30, a signal processing unit 32, an image composition unit 34, a display processing unit 36, a probe-position-and-posture information generation unit 38, a three-dimensional image data acquisition unit 40, and a position-and-posture measurement unit 48, and operates as an ultrasound image processing device.

The controller 30 executes the overall control of the ultrasound diagnostic apparatus 100. The controller 30 may acquire data obtained by each of the transmission-and-reception unit 12, the signal processing unit 32, the image composition unit 34, the display processing unit 36, the probe-position-and-posture information generation unit 38, and the three-dimensional image data acquisition unit 40. The controller 30 may execute information processing on the acquired data and output the data, which has been subjected to the information processing, to each of the transmission-and-reception unit 12, the signal processing unit 32, the image composition unit 34, the display processing unit 36, the probe-position-and-posture information generation unit 38, and the three-dimensional image data acquisition unit 40. In this manner, the controller 30 may execute the information processing in cooperation with each of the transmission-and-reception unit 12, the signal processing unit 32, the image composition unit 34, and the display processing unit 36.

The operation device 42 may comprise a button, a lever, a keyboard, a mouse, and the like. The operation device 42 may be a touch panel provided on the display unit 44. The controller 30 may execute the control of the ultrasound diagnostic apparatus 100 based on an operation by a user. The display unit 44 may be a display such as a liquid-crystal display or an organic EL display.

In the present embodiment, each processing is executed by any computer. Moreover, any computer may execute these kinds of processing by a processor as hardware, a program as software, or a combination thereof. In such a case, the processor is configured to execute various kinds of processing in the present embodiment in cooperation with the program, and may function as each unit or each means in the present embodiment. Further, the execution order of the processing by the processor is not limited to the above-described order, and may be changed as appropriate. Any computer may be a general-purpose computer, a computer for specific use, a workstation, or another system that can execute each processing.

The processor may be configured by one or more kinds of hardware, and the type of hardware is not limited. For example, the processor may be configured by a programmable logic device such as a central processing unit (CPU), a micro processing unit (MPU), or a field programmable gate array (FPGA), a dedicated circuit for executing specific processing, such as an application-specific integrated circuit (ASIC), or hardware such as a graphics processing unit (GPU) or a neural processing unit (NPU). Moreover, the type of hardware may be a combination of different kinds of hardware. In a case in which the plurality of kinds of hardware are configured to execute one or more kinds of processing of a certain processor, the plurality of kinds of hardware may be present in devices physically separated from each other or may be present in the same device. In addition, in any embodiment, the order of each processing by the processor is not limited to the order described in the specification of the present application, and may be changed as appropriate. In addition, hardware is implemented in a form of an electric circuit (circuitry) in which circuit elements, such as semiconductor elements, are combined.

Furthermore, the program may be software such as firmware or a microcode. The program may be, for example, a group of program modules, and each function thereof may be implemented by a processor configured to execute each function. The program may be a program code or a plurality of code segments stored in one or more non-transitory computer-readable media (for example, a storage medium and other storages). The program may be stored in the plurality of non-transitory computer-readable media present in devices physically separated from each other. The program code or the code segment may represent any combination of procedures, functions, subprograms, routines, subroutines, modules, software packages, classes, instructions, data structures, or program statements. The program code or the code segment may be connected to another code segment or a hardware circuit by transmitting and receiving information, data, arguments, parameters, or contents in the memory.

Each of the ultrasound probes 14a and 14b comprises a plurality of ultrasound transducers. Since the functions of the ultrasound probes 14a and 14b are the same as each other, the operation of the ultrasound probe 14a will be described here.

The transmission-and-reception unit 12 outputs a transmission signal, which is an electrical signal, to the plurality of ultrasound transducers provided in the ultrasound probe 14a. Each of the ultrasound transducers converts the transmission signal into the ultrasound, and transmits the ultrasound to the subject. The transmission-and-reception unit 12 adjusts a delay time of the transmission signal output to each of the ultrasound transducers, to form an ultrasound beam directed in a specific direction.

Each of the ultrasound transducers receives the ultrasound reflected in the subject, converts the ultrasound into the reception signal which is an electrical signal, and outputs the reception signal to the signal processing unit 32. The signal processing unit 32 adjusts the delay time of the reception signal output from each of the ultrasound transducers so that the reception signals of the ultrasound coming from a direction in which the ultrasound beam is directed are constructively added, and adds up the reception signals after the delay time adjustment. The signal processing unit 32 outputs a phase-aligned summed signal generated in this manner to the image composition unit 34.

Further, the transmission-and-reception unit 12 varies the delay time of the transmission signal output to each of the ultrasound transducers so that the inside of a specific observation cross section of the subject is scanned with the ultrasound beam. In addition, the signal processing unit 32 varies the delay time of the reception signal output from each of the ultrasound transducers so that the phase-aligned summed signal corresponding to the direction in which the inside of the subject is scanned with the ultrasound beam is generated, and adds up the reception signals after the delay time adjustment.

The image composition unit 34 generates B-mode image data as the display image data based on the phase-aligned summed signals acquired in each direction in the observation cross section, and outputs the B-mode image data to the display processing unit 36. The transmission-and-reception unit 12, the signal processing unit 32, and the image composition unit 34 generate the B-mode image data as the display image data in sequence with the elapse of time at a predetermined frame rate, and output the B-mode image data to the display processing unit 36. Here, the frame rate means the number of images generated per unit time.

The display processing unit 36 converts the display image data generated in sequence with the elapse of time into a video signal, and outputs the video signal to the display unit 44. The display unit 44 displays an image based on the display image data generated in sequence with the elapse of time, that is, a real-time image of a B-mode image, based on the video signal.

In the above description, the processing of scanning the inside of the subject with the ultrasound beam, generating the B-mode image data based on the phase-aligned summed signal corresponding to the ultrasound beam in each direction, and displaying the B-mode image has been described. The ultrasound diagnostic apparatus 100 may execute a Doppler mode operation in which a blood flow velocity is obtained based on a frequency shift (Doppler shift) of the phase-aligned summed signal from a frequency of the transmission signal.

The image composition unit 34 obtains, for example, the blood flow velocity in a predetermined range on the ultrasound beam in each direction, and generates color Doppler data showing a blood flow velocity distribution on the observation cross section. The color Doppler data indicates the blood flow velocity distribution on the observation cross section by indicating a region in which blood flows in a direction toward the ultrasound probe 14a and a region in which blood is separated from the ultrasound probe 14a in different colors. The image composition unit 34 generates color Doppler B-mode image data in which the B-mode image is colored to indicate the blood flow velocity distribution, as the display image data, and outputs the display image data to the display processing unit 36.

The display processing unit 36 converts the display image data generated in sequence with the elapse of time into a video signal, and outputs the video signal to the display unit 44. The display unit 44 displays an image based on the display image data generated in sequence with the elapse of time, that is, a real-time image of a color Doppler B-mode image, based on the video signal.

In the above description, the processing has been described in which the transmission-and-reception unit 12 transmits and receives the ultrasound by the ultrasound probe 14a, and the signal processing unit 32 and the image composition unit 34 generate the ultrasound image data, such as the B-mode image data, the color Doppler data, and the color Doppler B-mode image data. Through the same processing, the transmission-and-reception unit 12 may transmit and receive the ultrasound by the ultrasound probe 14b, and the signal processing unit 32 and the image composition unit 34 may generate the ultrasound image data. In addition, the transmission-and-reception unit 12, the signal processing unit 32, and the image composition unit 34 may alternately generate the ultrasound image data obtained by the ultrasound probe 14a and the ultrasound image data obtained by the ultrasound probe 14b in a time-division manner, for example, with the elapse of time.

In this case, the display processing unit 36 may generate the video signal such that an image based on the ultrasound image data obtained by the ultrasound probe 14a and an image based on the ultrasound image data obtained by the ultrasound probe 14b are displayed on the display unit 44 in sequence with the elapse of time, and output the video signal to the display unit 44. The display unit 44 displays the image based on the ultrasound image data obtained by the ultrasound probe 14a and the image based on the ultrasound image data obtained by the ultrasound probe 14b in real time, based on the video signal.

In addition, the image displayed by the display unit 44 may be a still image. In such a case, the image composition unit 34 outputs, for example, the display image data of one frame to the display processing unit 36. The display processing unit 36 generates a video signal for displaying the image based on the display image data of one frame as the still image, and outputs the video signal to the display unit 44. The display unit 44 displays the image based on the display image data as the still image based on the video signal.

In the following description, each image displayed by the display unit 44 may also be the still image or the real-time image. In a case in which the display unit 44 displays the real-time image, the image composition unit 34 generates the display image data in sequence with the elapse of time based on various kinds of data acquired in sequence with the elapse of time by the signal processing unit 32, and outputs the display image data to the display processing unit 36. The display processing unit 36 converts the display image data generated in sequence with the elapse of time into a video signal, and outputs the video signal to the display unit 44. The display unit 44 displays the real-time image based on the display image data generated in sequence with the elapse of time based on the video signal.

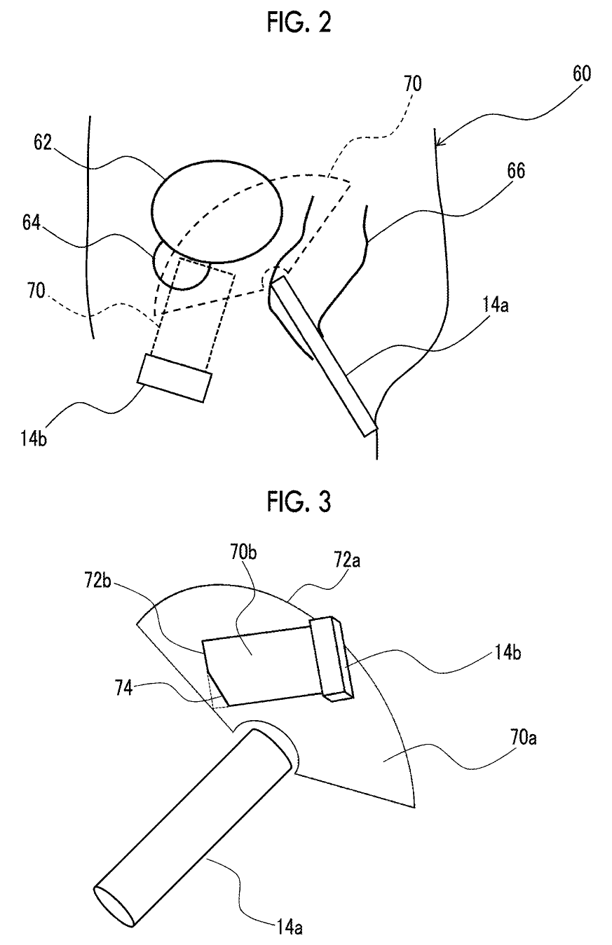

FIG. 2 shows an example of diagnosis performed using the ultrasound diagnostic apparatus 100. FIG. 2 shows a bladder 62, a prostate 64, and a rectum 66 of a subject 60. In this example, the ultrasound probe 14a is a transrectal probe, and is inserted into the rectum 66. The ultrasound probe 14b is a linear probe, and is disposed such that a distal end surface for the ultrasound transmission and reception is in contact with a body surface of the subject 60.

In the example shown in FIG. 2, the ultrasound probe 14a performs a convex scan with the ultrasound beam in the observation cross section that crosses the prostate 64. Here, the convex scan refers to a scan with the ultrasound beam radiated from a curved surface, for the ultrasound transmission and reception, along the curved surface. In addition, the ultrasound probe 14b performs a linear scan with the ultrasound beam in the observation cross section that crosses the prostate 64. Here, the linear scan refers to a scan with the ultrasound beam radiated from a surface for the ultrasound transmission and reception, in a straight line while keeping a direction constant. In the example shown in FIG. 2, the observation cross section of the ultrasound probe 14a and the observation cross section of the ultrasound probe 14b intersect each other.

An operator may perform, for example, a surgical operation of resecting the prostate 64 while leaving the neurovascular bundle based on the ultrasound image in the observation cross section of the ultrasound probe 14a and the ultrasound image in the observation cross section of the ultrasound probe 14b.

FIG. 3 is a diagram schematically showing a probe positional relationship diagram showing an example of a position and a posture of each of the ultrasound probes 14a and 14b. In addition, a first observation plane 70a on which the convex scan with the ultrasound beam is performed by the ultrasound probe 14a and a second observation plane 70b on which the linear scan with the ultrasound beam is performed by the ultrasound probe 14b are shown. The maximum depths, which can be observed, of the first observation plane 70a and the second observation plane 70b are indicated by a depth boundary edge 72a and a depth boundary edge 72b, respectively. A region at one corner of the deepest portion of a rectangular second observation plane 70b penetrates the first observation plane 70a. In FIG. 3, an intersection line 74 between the first observation plane 70a and the second observation plane 70b is indicated by a solid line.

The operator performs the surgical operation on the prostate 64 while referring to the B-mode image displayed by the ultrasound probe 14a, which is the transrectal probe, and the B-mode image displayed by the ultrasound probe 14b, which is the linear probe.

In the ultrasound diagnostic apparatus 100 according to the present embodiment, an intersection line/convex scan image in which the intersection line 74 is drawn on the B-mode image displayed by the ultrasound probe 14a is displayed on the display unit 44. Processing of displaying the intersection line/convex scan image will be described again with reference to FIG. 1.

The positioning signal generator 58 is connected to the position-and-posture measurement unit 48, and the positioning signal generator 58 is caused to transmit a positioning signal. The positioning signal transmitted from the positioning signal generator 58 may be any of a radio wave, ultrasound, or a magnetic field. The ultrasound probes 14a and 14b are provided with position-and-posture sensors 20a and 20b, respectively. The position-and-posture sensor 20a receives the positioning signal, generates a detection signal for measuring the position and the posture of the ultrasound probe 14a (hereinafter, referred to as position and posture), and outputs the detection signal to the position-and-posture measurement unit 48. Similarly, the position-and-posture sensor 20b receives the positioning signal, generates a detection signal for measuring the position and the posture of the ultrasound probe 14b, and outputs the detection signal to the position-and-posture measurement unit 48.

The position of the ultrasound probe 14a is a position of a reference point defined in the ultrasound probe 14a. The posture of the ultrasound probe 14a may be defined by an angle between a reference straight line defined in the ultrasound probe 14a and an xy plane of an xyz coordinate system fixed to the ultrasound diagnostic apparatus 100, an angle between the reference straight line and a z-axis, or the like. The reference straight line may be defined, for example, based on a direction in which the plurality of ultrasound transducers are arranged. As in the ultrasound probe 14a, the position and the posture of the ultrasound probe 14b may also be defined by the reference point and the reference straight line.

The position-and-posture measurement unit 48 generates position-and-posture information indicating the position and the posture of the ultrasound probe 14a based on the detection signal output as the output value from the position-and-posture sensor 20a and the positioning signal output to the positioning signal generator 58, and outputs the generated position-and-posture information to the probe-position-and-posture information generation unit 38. Similarly, the position-and-posture measurement unit 48 generates position-and-posture information indicating the position and the posture of the ultrasound probe 14b based on the detection signal output as the output value from the position-and-posture sensor 20b and the positioning signal output to the positioning signal generator 58, and outputs the generated position-and-posture information to the probe-position-and-posture information generation unit 38.

The probe-position-and-posture information generation unit 38 acquires scan range information indicating the scan range of the ultrasound beam for the ultrasound probes 14a and 14b from the controller 30. The scan range information of each of the ultrasound probes 14a and 14b indicates the scan range with respect to the coordinate system fixed to each of the ultrasound probes 14a and 14b. The probe-position-and-posture information generation unit 38 obtains intersection line information indicating a range in which the intersection line 74 shown in FIG. 3 extends in an xyz three-dimensional space based on the scan range information of each of the ultrasound probes 14a and 14b and the position-and-posture information of each of the ultrasound probes 14a and 14b. The intersection line information includes, for example, a constant that defines a function z = f(x, y) defined in the xyz coordinate system. The probe-position-and-posture information generation unit 38 outputs the intersection line information and the position-and-posture information of the ultrasound probes 14a and 14b to the controller 30.

The controller 30 outputs the intersection line information, the scan range information for the ultrasound probes 14a and 14b, and the position-and-posture information for the ultrasound probes 14a and 14b to the image composition unit 34. The image composition unit 34 generates intersection line/convex scan image data based on the B-mode image data obtained by the ultrasound probe 14a, the intersection line information, and the scan range information and the position-and-posture information of the ultrasound probes 14a and 14b.

As described above, in the ultrasound diagnostic apparatus 100 according to the present embodiment, the information processing unit 10 generates the position-and-posture information of each of the ultrasound probes 14a and 14b based on the output values of the position-and-posture sensors 20a and 20b provided in the ultrasound probes 14a and 14b, respectively, and generates the intersection line/convex scan image data as the display image data based on the ultrasound image data of each of the ultrasound probes 14a and 14b, the position-and-posture information of one of the ultrasound probe 14a or the ultrasound probe 14b, and the position-and-posture information of the other ultrasound probe.

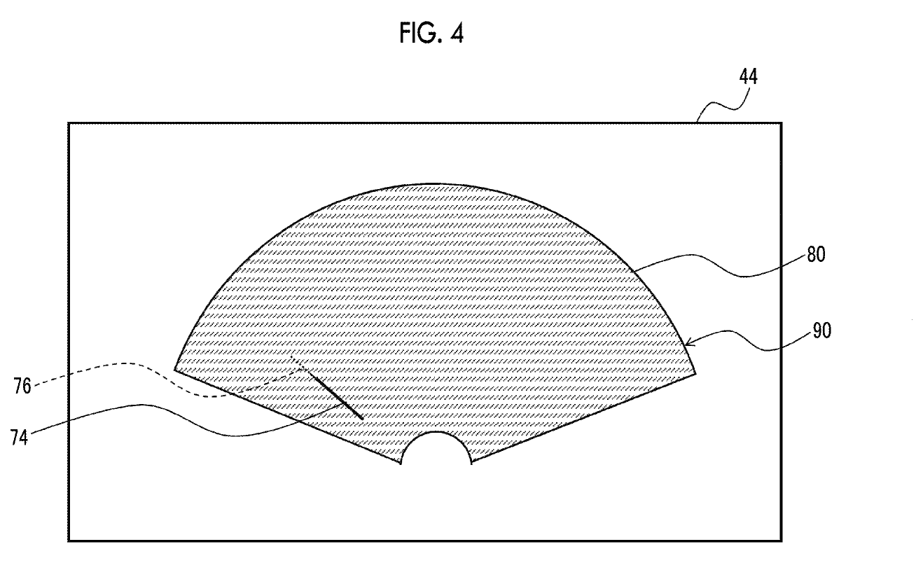

FIG. 4 shows an example of an intersection line/convex scan image 90 displayed on the display unit 44. In the example shown in this drawing, the intersection line 74 is indicated by a solid line on a B-mode image 80 obtained by the ultrasound probe 14a. In addition to the intersection line 74, an extension line 76 (extended line) obtained by extending the intersection line 74 is indicated by a broken line.

The intersection line 74 and the extension line 76 displayed on the display unit 44 are probe indicators indicating an image (hereinafter, may be referred to as an ultrasound image) shown by the ultrasound image data corresponding to one of the ultrasound probes 14a and 14b and the ultrasound transmission range of the other ultrasound probe.

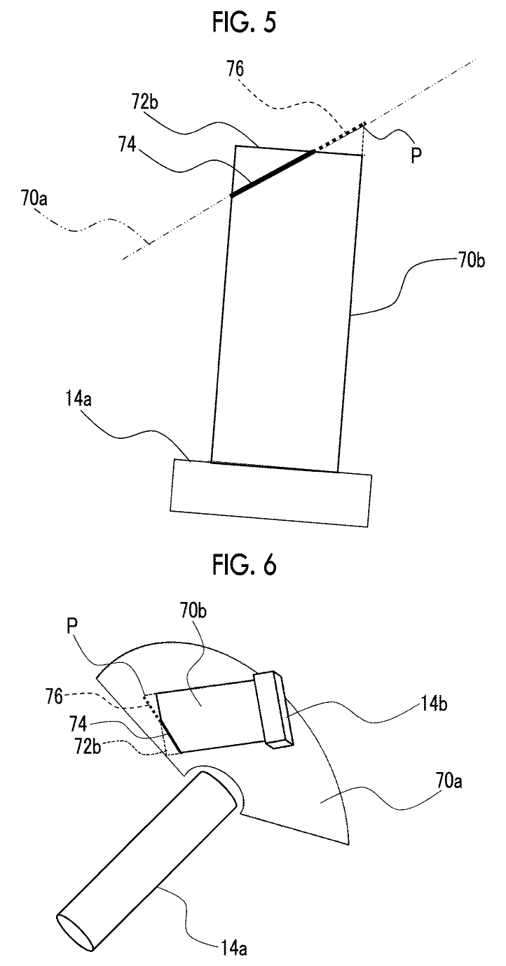

FIG. 5 shows a diagram of the first observation plane 70a and the second observation plane 70b in a direction being perpendicular to a normal line of the first observation plane 70a and viewing a plane on which the second observation plane 70b extends. A corner portion formed at the left end of the depth boundary edge 72b of the second observation plane 70b penetrates the first observation plane 70a and is located in a region deeper than the first observation plane 70a. The extension line 76 further extends from an end point in a direction from a start point on a depth direction edge on the penetration side of the second observation plane 70b to the end point on the depth boundary edge 72b. The depth direction edge on the penetration side refers to one edge that penetrates the first observation plane 70a among a pair of depth direction edges of the first observation plane 70a.

The extension line 76 has the end point of the intersection line 74 as the start point and has a projection point P, which is formed at the intersection of the first observation plane 70a and a straight line extending in the depth direction from the depth direction edge of the non-penetration side, as the end point. That is, the extension line 76 is a straight line connecting the end point of the intersection line 74 to the projection point P.

The probe indicator may be the intersection line 74 between the scan range of the ultrasound beam formed by the ultrasound probe 14a (first ultrasound probe) and the scan range of the ultrasound beam formed by the ultrasound probe 14b (second ultrasound probe), and the extension line 76 obtained by extending the intersection line 74. The extension line 76 is a projection image in which the scan range of the ultrasound beam formed by the ultrasound probe 14b is projected onto the ultrasound image obtained by the ultrasound probe 14a. The projection image is a projection diagram in a case in which a scan range of an ultrasound beam formed by the ultrasound probe 14b is projected onto the ultrasound image formed by the ultrasound probe 14a along a direction of an ultrasound beam formed by the second ultrasound probe.

FIG. 6 is a diagram in which an extension line 76 is added to the probe positional relationship diagram shown in FIG. 3. The extension line 76 extends from the end point of the intersection line 74 in a direction in which the intersection line 74 extends, and the extension line 76 ends at the projection point P at which the first observation plane 70a intersects the straight line obtained by extending the depth direction edge on the non-penetration side in the depth direction.

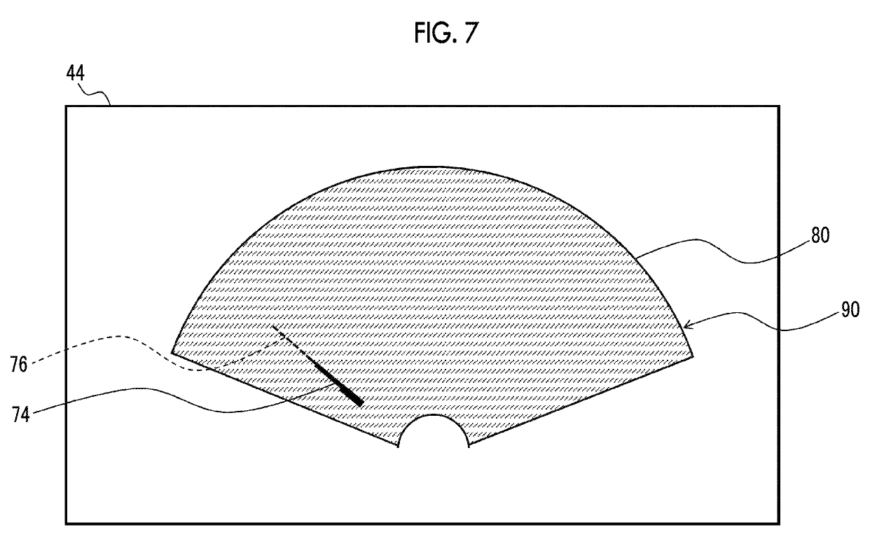

The image composition unit 34 may generate the intersection line/convex scan image data such that the intersection line 74 and the extension line 76 are drawn in various aspects. For example, as shown in FIG. 7, the image composition unit 34 may generate the intersection line/convex scan image data such that a section of the intersection line 74 on the end point side is drawn with a thinner solid line than the start point side, and the extension line 76 on a side away from the intersection line 74 with respect to the section drawn with the solid line is drawn with a broken line. In addition, the intersection line 74 may be drawn such that the line thickness decreases from the start point toward the end point. In addition, the intersection line 74 may be drawn such that the color varies from the start point toward the end point. The extension line 76 may also be drawn such that the color varies as the distance from the end point of the intersection line 74 increases.

In addition, the image composition unit 34 may generate intersection line/linear scan image data based on the B-mode image data obtained by the ultrasound probe 14b, the intersection line information, and the scan range information and the position-and-posture information of the ultrasound probes 14a and 14b. The intersection line/linear scan image data indicates an image (intersection line/linear scan image) in which the intersection line 74 is drawn on the B-mode image displayed by the ultrasound probe 14b.

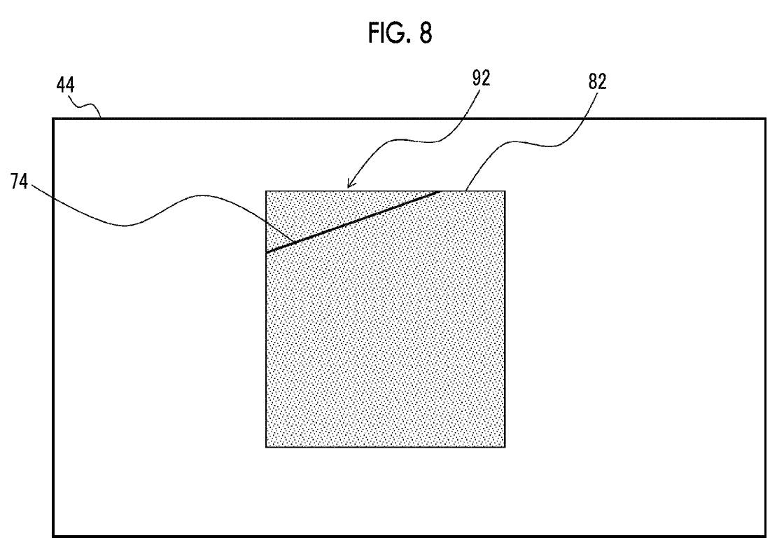

FIG. 8 shows an example of an intersection line/linear scan image 92 displayed on the display unit 44. In the intersection line/linear scan image 92, the intersection line 74 is drawn on the B-mode image 82 by the ultrasound probe 14b. In this drawing, an up direction corresponds to the depth direction. A left vertical edge of the intersection line/linear scan image 92 corresponds to the depth direction edge of the second observation plane 70b on the penetration side, and a right vertical edge thereof corresponds to the depth direction edge of the second observation plane 70b on the non-penetration side. An upper horizontal edge corresponds to the depth boundary edge 72b of the second observation plane 70b. The intersection line 74 extends from the start point on the left vertical edge to the end point on the upper horizontal edge.

With the ultrasound diagnostic apparatus 100 according to the present embodiment, the intersection line/convex scan image 90 shown in FIG. 4 or FIG. 7 or the intersection line/linear scan image 92 shown in FIG. 8 is displayed on the display unit 44. That is, the image in which the intersection line 74 is drawn on the B-mode image is displayed on the display unit 44. For the intersection line/convex scan image 90, the extension line 76 is drawn on the B-mode image. Therefore, in a case in which the subject can be observed from different directions using the two ultrasound probes 14a and 14b, the user can easily grasp the positional relationship between the first observation plane 70a and the second observation plane 70b by the intersection line 74 and the extension line 76.

The image composition unit 34 may generate the display image data showing the image in which the intersection line/convex scan image 90 and the intersection line/linear scan image 92 are arranged, and output the generated display image data to the display processing unit 36. That is, the image composition unit 34 may generate the display image data showing the image in which the intersection line/convex scan image 90 and the intersection line/linear scan image 92 are arranged, based on the B-mode image data obtained by the ultrasound probe 14a, the B-mode image data obtained by the ultrasound probe 14b, the intersection line information, and the scan range information and the position-and-posture information of the ultrasound probes 14a and 14b, and output the generated display image data to the display processing unit 36. The display processing unit 36 converts the display image data into the video signal, and outputs the converted video signal to the display unit 44. The display unit 44 displays the image based on the display image data, that is, the image in which the intersection line/convex scan image 90 and the intersection line/linear scan image 92 are arranged, based on the video signal.

The image composition unit 34 may generate the probe positional relationship image data showing the probe positional relationship diagram as shown in FIG. 3 or FIG. 6. In this case, the image composition unit 34 generates the probe positional relationship image data as the display image data based on the intersection line information and the scan range information and the position-and-posture information of the ultrasound probes 14a and 14b.

The display processing unit 36 converts the display image data into the video signal, and outputs the converted video signal to the display unit 44. The display unit 44 displays the image based on the display image data, that is, the probe positional relationship image based on the video signal. The probe positional relationship diagram is drawn on the probe positional relationship image.

The display processing unit 36 may generate the display image data showing the probe positional relationship image in parallel with at least one of the intersection line/convex scan image 90 or the intersection line/linear scan image 92 or showing the image in which the probe positional relationship image is superimposed on one of the intersection line/convex scan image 90 or the intersection line/linear scan image 92. The image composition unit 34 generates the display image data based on the intersection line information and the scan range information and the position-and-posture information of the ultrasound probes 14a and 14b, and outputs the generated display image data to the display processing unit 36. The display processing unit 36 converts the display image data into the video signal and outputs the converted video signal to the display unit 44. The display unit 44 displays the image based on the display image data, that is, the probe positional relationship image based on the video signal.

The display processing unit 36 may display the three-dimensional probe positional relationship image in which the probe positional relationship diagram is superimposed on the three-dimensional image obtained by an external apparatus such as a CT or MRI apparatus, on the display unit 44. The three-dimensional image data acquisition unit 40 shown in FIG. 1 reads the three-dimensional image data from the external apparatus and outputs the read three-dimensional image data to the controller 30. The controller 30 outputs the three-dimensional image data to the image composition unit 34.

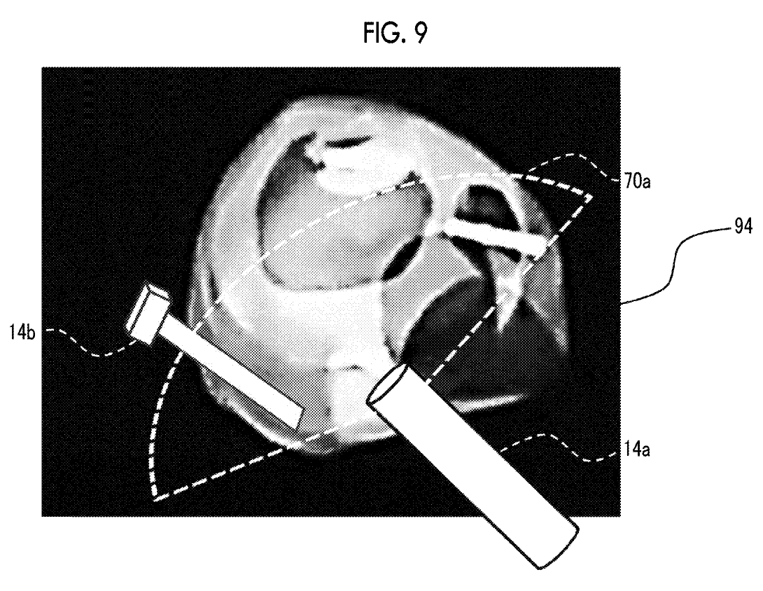

The image composition unit 34 generates the three-dimensional probe positional relationship image data as the display image data based on the intersection line information, the scan range information of the ultrasound probes 14a and 14b, and the three-dimensional image data, and outputs the generated three-dimensional probe positional relationship image data to the display processing unit 36. The display processing unit 36 converts the display image data into the video signal and outputs the converted video signal to the display unit 44. The display unit 44 displays the image based on the display image data, that is, the three-dimensional probe positional relationship image based on the video signal. FIG. 9 shows an example of the three-dimensional probe positional relationship image 94.

The display processing unit 36 may generate the display image data showing the three-dimensional probe positional relationship image 94 in parallel with at least one of the intersection line/convex scan image 90 or the intersection line/linear scan image 92 or showing the image in which the probe positional relationship image is superimposed on one of the intersection line/convex scan image 90 or the intersection line/linear scan image 92. The image composition unit 34 generates such display image data based on the three-dimensional probe positional relationship image data and at least one of the intersection line/convex scan image data or the intersection line/linear scan image data, and outputs the generated display image data to the display processing unit 36. The display processing unit 36 converts the display image data into the video signal and outputs the converted video signal to the display unit 44. The display unit 44 displays the image based on the display image data based on the video signal.

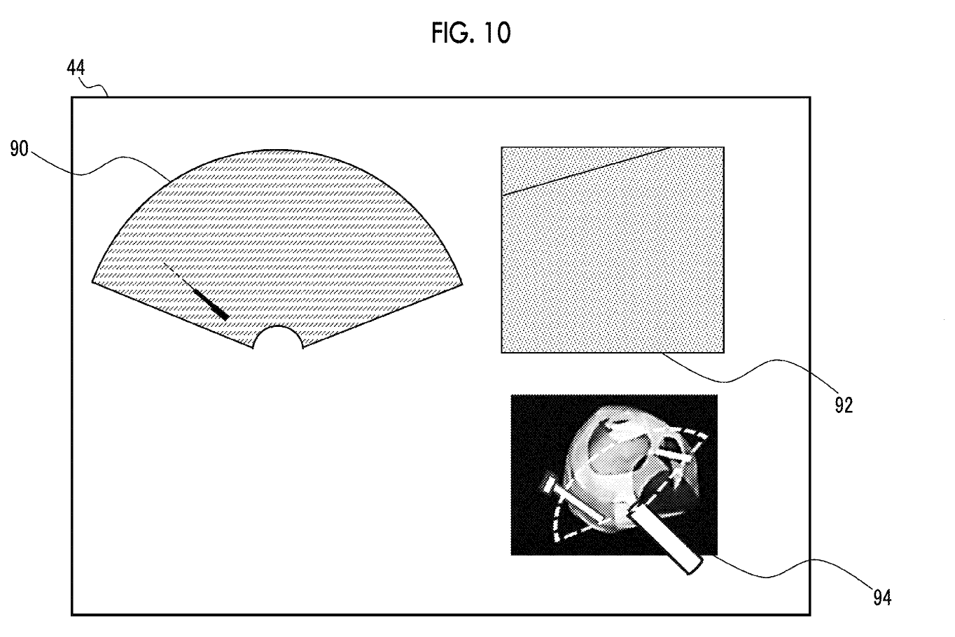

FIG. 10 shows the intersection line/convex scan image 90, the intersection line/linear scan image 92, and the three-dimensional probe positional relationship image 94 displayed side by side on the display unit 44. The intersection line/convex scan image 90 is displayed on a left side of an upper part, and the intersection line/linear scan image 92 is displayed on a right side of the upper part. The three-dimensional probe positional relationship image 94 is displayed in a lower part. The three-dimensional probe positional relationship image 94 displayed on the display unit 44 may be replaced with the probe positional relationship image showing the probe positional relationship diagram shown in FIG. 3 or FIG. 6.

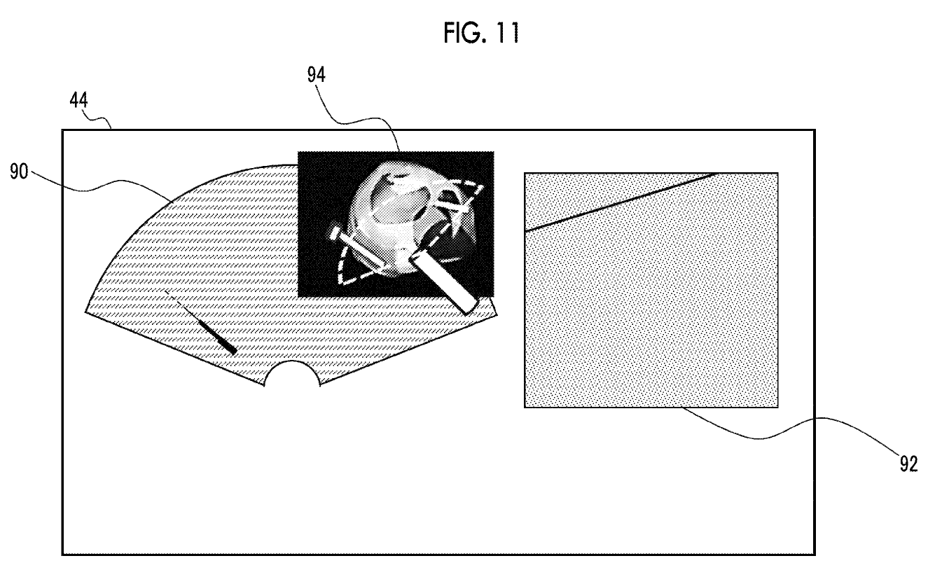

FIG. 11 shows the intersection line/convex scan image 90 and the intersection line/linear scan image 92 displayed side by side on the display unit 44, and the three-dimensional probe positional relationship image 94 displayed in a superimposed manner on the intersection line/convex scan image 90. The intersection line/convex scan image 90 is displayed on the left side, and the intersection line/linear scan image 92 is displayed on the right side. The three-dimensional probe positional relationship image 94 is displayed in a superimposed manner at the upper right of the intersection line/convex scan image 90. The three-dimensional probe positional relationship image 94 displayed on the display unit 44 may be replaced with the probe positional relationship image showing the probe positional relationship diagram shown in FIG. 3 or FIG. 6.

In the above description, the processing of generating the ultrasound image data showing the two-dimensional image in the image composition unit 34 has been described. The image composition unit 34 may generate volume data showing a set of voxels (pixels).

In this case, for the ultrasound probe 14a, for example, an ultrasound probe having a two-dimensional array structure in which the ultrasound transducers are arranged in two orthogonal axial directions (major axis direction and minor axis direction) is used. The controller 30, the transmission-and-reception unit 12, and the ultrasound probe 14a oscillate the observation plane in an azimuth angle direction in which the transmission beam and the reception beam intersect the observation plane that is a scanned. The oscillation of the observation plane may be performed around an axis in the major axis direction of the two-dimensional array structure.

The controller 30, the transmission-and-reception unit 12, and the ultrasound probe 14a cause the observation plane to oscillate in the azimuth angle direction by a predetermined step size each time the B-mode image data of one observation plane is generated, for example. The image composition unit 34 generates the B-mode image data of one observation plane each time the observation plane oscillates by the step size. The image composition unit 34 generates the volume data composed of the B-mode image data of a plurality of frames acquired at different azimuth angles. The volume data represents each pixel value of the plurality of voxels arranged in the three-dimensional space.

The image composition unit 34 may generate volume rendering image data representing a plurality of voxels arranged in the three-dimensional space as a two-dimensional image, and output a volume rendering image data to the display processing unit 36. The display processing unit 36 generates the video signal for displaying the volume rendering image, and outputs the generated video signal to the display unit 44. The display unit 44 displays the volume rendering image based on the video signal.

In the above-described embodiment, the volume data is acquired by the controller 30, the transmission-and-reception unit 12, and the ultrasound probe 14a oscillating the observation plane. In addition to this processing, processing of acquiring the volume data by moving the ultrasound probe 14a in a linear or curved manner may be executed. The ultrasound probe 14a may be moved by the hand of the operator or by a mechanism that moves the ultrasound probe 14a.

Here, although the example has been described in which the volume data is generated by the ultrasound probe 14a, the volume data may be generated by the ultrasound probe 14b using the same configuration and processing.

In a case in which the volume data is acquired by one of the ultrasound probes 14a and 14b and the two-dimensional ultrasound image data is acquired by the other, a two-dimensional intersection region in which the three-dimensional shape corresponding to the volume data and the observation plane intersect each other is displayed on the display unit 44 instead of the intersection line 74. The two-dimensional intersection region is planar. In addition, in a case in which the volume data is acquired by both the ultrasound probes 14a and 14b, a three-dimensional intersection region in which two three-dimensional bodies corresponding to the two pieces of volume data intersect with each other is displayed on the display unit 44 instead of the intersection line 74. The three-dimensional intersection region is volumetric.

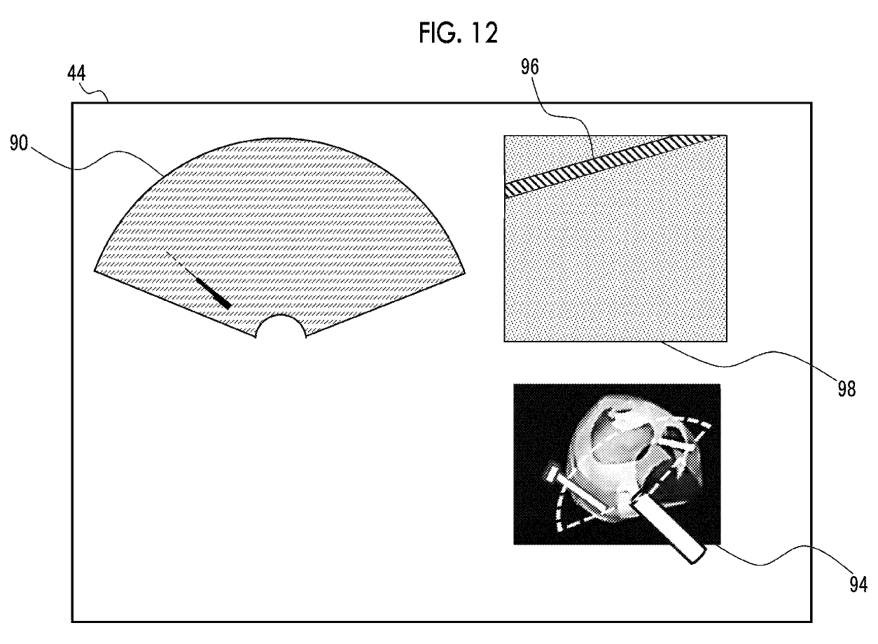

FIG. 12 shows an example of an image displayed on the display unit 44 in a case in which the volume data is acquired by the ultrasound probe 14a which is a transrectal probe and the two-dimensional ultrasound image data is acquired by the ultrasound probe 14b which is a linear probe. That is, FIG. 12 shows the intersection line/convex scan image 90, a two-dimensional intersection region/linear scan image 98, and the three-dimensional probe positional relationship image 94 displayed side by side on the display unit 44. The intersection line/convex scan image 90 is displayed on a left side of an upper part, and the two-dimensional intersection region/linear scan image 98 is displayed on the right side of the upper part. The three-dimensional probe positional relationship image 94 is displayed in a lower part. The two-dimensional intersection region/linear scan image 98 is an image in which a figure indicating the two-dimensional intersection region is superimposed on the B-mode image.

The probe indicator displayed by the ultrasound diagnostic apparatus 100 according to the present embodiment indicates a region in which the scan range of the ultrasound beam formed by the ultrasound probe 14a (first ultrasound probe which is one of the plurality of ultrasound probes) and the scan range of the ultrasound beam formed by the ultrasound probe 14b (second ultrasound probe which is the other ultrasound probe) intersect each other. The intersection line 74 and the extension line 76 shown in FIGS. 4 and 7, and the intersection line 74 shown in FIG. 8 are the probe indicators. In addition, the two-dimensional intersection region 96 shown in the two-dimensional intersection region/linear scan image 98 is the probe indicator. The three-dimensional intersection region displayed in a case in which the volume data is acquired by both the ultrasound probes 14a and 14b is also the probe indicator.

The present disclosure is also applicable to a program and a program product. The above-described embodiment of the present disclosure relates to an ultrasound image processing program read by an ultrasound image processing device, the program causing a processor provided in the ultrasound image processing device to execute a process comprising: generating ultrasound image data corresponding to each of a plurality of ultrasound probes based on a reception signal output from each of the ultrasound probes; acquiring position-and-posture information of each of the ultrasound probes; and generating display image data showing an ultrasound image obtained by one of the plurality of ultrasound probes and a probe indicator indicating an ultrasound transmission range of the other ultrasound probe, based on the ultrasound image data corresponding to each of the ultrasound probes and the position-and-posture information. The ultrasound image processing program may be provided by being stored in a storage medium, such as a memory card, a USB memory, or a CD-ROM.

In the above description, the embodiment has been described in which the convex scan is performed in the ultrasound probe 14a, and the linear scan is performed in the ultrasound probe 14b. The scan with the ultrasound beam in the ultrasound probes 14a and 14b may be performed by other methods. For example, a radial scan in which the ultrasound probe performs a scan with the ultrasound beam in a donut shaped pattern, or a sector scan in which the ultrasound probe performs a scan with the ultrasound beam in a fan-shaped pattern with a reference point in the ultrasound probe as a center may be performed.

In the above description, the embodiment used for the surgical operation on the prostate has been described. The present invention may also be applied to other surgical operations. For example, the esophagus surgery may be performed while observing two B-mode images obtained from the esophagus and the body surface, with the ultrasound probe 14a as the esophageal probe and the ultrasound probe 14b as the intraoperative probe such as the linear probe.

What is claimed is:

1. An ultrasound image processing device comprising:
a processor configured to:
generate ultrasound image data corresponding to each of a plurality of ultrasound probes based on a reception signal output from each of the ultrasound probes;
acquire position-and-posture information of each of the ultrasound probes; and
generate display image data showing an ultrasound image obtained by one of the plurality of ultrasound probes and a probe indicator indicating an ultrasound transmission range of the other ultrasound probe, based on the ultrasound image data corresponding to each of the ultrasound probes and the position-and-posture information.

2. The ultrasound image processing device according to claim 1,
wherein the probe indicator indicates a region in which a scan range of an ultrasound beam formed by a first ultrasound probe, which is one of the plurality of ultrasound probes, and a scan range of an ultrasound beam formed by a second ultrasound probe, which is the other ultrasound probe, intersect each other.

3. The ultrasound image processing device according to claim 2,
wherein the probe indicator includes an intersection line between the scan range of the ultrasound beam formed by the first ultrasound probe and the scan range of the ultrasound beam formed by the second ultrasound probe, and an extension line formed by extending the intersection line, and
the extension line extends in accordance with a projection image in which the scan range of the ultrasound beam formed by the second ultrasound probe is projected onto an ultrasound image obtained by the first ultrasound probe in a direction of the ultrasound beam formed by the second ultrasound probe.

4. The ultrasound image processing device according to claim 2,
wherein the display image data is image data in which the probe indicator is displayed in a superimposed manner on any of an ultrasound image obtained by the first ultrasound probe or an ultrasound image obtained by the second ultrasound probe.

5. The ultrasound image processing device according to claim 1,
wherein the display image data includes a probe positional relationship image showing a scan range of an ultrasound beam formed by a first ultrasound probe, which is one of the plurality of ultrasound probes, a scan range of an ultrasound beam formed by a second ultrasound probe, which is the other ultrasound probe, and a positional relationship between the first ultrasound probe and the second ultrasound probe.

6. An ultrasound diagnostic apparatus comprising:
the ultrasound image processing device according to claim 1;
each of the ultrasound probes; and
a position-and-posture sensor provided in each of the ultrasound probes,
wherein the processor is configured to:
generate the position-and-posture information of each of the ultrasound probes based on an output value of the position-and-posture sensor provided in each of the ultrasound probes; and
generate the display image data based on the position-and-posture information of one of the plurality of ultrasound probes and the position-and-posture information of the other ultrasound probe.

7. A non-transitory computer-readable storage medium storing an ultrasound image processing program read by an ultrasound image processing device, the program causing a processor provided in the ultrasound image processing device to execute a process comprising:
generating ultrasound image data corresponding to each of a plurality of ultrasound probes based on a reception signal output from each of the ultrasound probes;
acquiring position-and-posture information of each of the ultrasound probes; and
generating display image data showing an ultrasound image obtained by one of the plurality of ultrasound probes and a probe indicator indicating an ultrasound transmission range of the other ultrasound probe, based on the ultrasound image data corresponding to each of the ultrasound probes and the position-and-posture information.
